(19) United States
(12) Patent Application Publication
(10) Pub. No.: US 2015/0265704 A1
(43) Pub. Date: Sep. 24, 2015

(54) IGM THERAPY IN PREVENTION OF ONSET, PROGRESSION, AND RECURRENCE OF AUTOIMMUNE TYPE 1 DIABETES

(71) Applicant: UNIVERSITY OF VIRGINIA PATENT FOUNDATION, Charlottesville, VA (US)

(72) Inventors: Kenneth Brayman, Charlottesville, VA (US); Preeti Chhabra, Waynesboro, VA (US); Peter I. Lobo, Charlottesville, VA (US)

(73) Assignee: UNIVERSITY OF VIRGINIA PATENT FOUNDATION, Charlottesville, VA (US)

(21) Appl. No.: 14/434,179

(22) PCT Filed: Oct. 8, 2013
(86) PCT No.: PCT/US2013/063839

§ 371 Date: Apr. 8, 2015

Related U.S. Application Data
(60) Provisional application No. 61/710,931, filed on Oct. 8, 2012.

(51) Int. Cl. A61K 39/395; A61K 35/39; A61K 45/06; C07K 16/06

(52) U.S. Cl. CPC A61K 39/39516; C07K 16/06; A61K 35/39; A61K 45/06

(57) ABSTRACT

The therapeutic potential of polyclonal serum naturally occurring IgM (nIgM) administration in preventing the onset and progression of autoimmune type 1 diabetes and in promoting graft survival following islet allotransplantation has been investigated. nIgM therapy prevents both, onset and progression of diabetes and promotes islet graft survival.

### CROSS REFERENCE TO RELATED APPLICATIONS

This application is entitled to priority pursuant to 35 U.S.C. §119(e) to U.S. provisional patent application no. 61/710,931, filed Oct. 8, 2012. The entire disclosure of the afore-mentioned patent application is incorporated herein by reference.

### BACKGROUND

Autoimmune type 1 diabetes mellitus (T1D) is a multifactorial disease caused by the progressive destruction of insulin-producing pancreatic β-cells that results in an absolute loss of insulin production [1-2]. Prevention or reversal of T1D either by preventing or arresting autoimmunity or through restoration of beta-cell mass and function is the ultimate goal of clinical intervention. Diagnosis of the disease in the latter stages of prehyperglycemic beta-cell destruction restricts the types of immunotherapies that can be implemented. To date, pancreas and islet transplantation remain the only reliable cures of diabetes that have met with consistent success [3-4]. However, the requisite chronic immunosuppressive therapy with its deleterious diabetogenic and nephrotoxic side-effects, such as the risk associated with global deletion of T cells and the development of infections and malignancies, remains the Achilles heel of therapeutic interventions aimed at improving islet graft survival and preventing autoimmune diabetes [5-6]. Another major drawback of chronic immunosuppressive therapy is its ineffectiveness over the long term that results in recurrence of disease upon drug withdrawal. Therefore, the development of novel therapeutic interventions that can prevent both autoimmune and alloimmune-mediated beta cell destruction by establishing effective long-term immunological unresponsiveness in the absence of chronic immunosuppression, with minimal or no deleterious side effects, is the principal focus of ongoing multi-disciplinary research. Current efforts to this end have not met with much clinical success.

Repertoires of naturally-occurring autoantibodies have been intensively studied during the last three decades and thought to play a significant role in the immunology of health and disease. These auto-antibodies are predominantly of the IgM isotype and can be found in umbilical cord blood along with the long lived, self-renewing B-1 lymphocytes that produce them, prior to exposure to foreign Ags; hence, referred to as naturally-occurring or natural IgM (nIgM) [7-8]. nIgMs and a subset known as nIgM anti-leukocyte autoantibodies (nIgM-ALAs) are present at low levels in normal individuals and increase during inflammatory disorders and various infections [9]. Previous studies in our laboratory and others have demonstrated that polyclonal serum nIgM and nIgM-ALA are a heterogeneous group of antibodies that are reactive to autologous and allogeneic cells with specificities for different, largely undefined membrane receptors that include receptors with phospholipids, glycolipids and glycoproteins as well as other cells that express leukocyte receptors [7-10]. Encoded by minimally or nonmutated germline genes, nIgMs are characteristically polyreactive with low binding affinity. Of particular importance, these autoantibodies do not mediate cytolysis in the presence of complement at body temperature and differ from disease-producing autoantibodies in that the latter are predominantly of the IgG isotype that bind with high affinity and specificity to autoantigens and mediate cytolysis at 37° C.

Elevated levels of nIgM-ALAs have been associated with lower incidence and severity of acute human heart and kidney graft rejections, thus permitting better graft survival [10-12]. We have previously shown that nIgM-ALA plays a regulatory role in attenuating inflammation mediated by innate and adaptive immune mechanisms, including where the inflammatory response involved Th-17 cells that were not effectively regulated by regulatory T cells [10]. Additionally, purified human serum nIgM immunoprecipitated human CD3, CD4 T cells and chemokine receptors CCRS and CXCR4 from whole cell lysates, downregulated CD2, CD86 and CD4, inhibited leukocyte production of proinflammatory cytokines TNF-alpha, IL-13 and IL-2, inhibited T cell activation and proliferation as well as leukocyte chemotaxis induced by chemokines, indicating an innate mechanism that acts to down-regulate T cell mediated inflammatory responses [7-10]. Polyclonal nIgMs have also been shown to contribute to the maintenance of immune homeostasis and to prevent autoimmunity [13-14].

There is a long felt need in the art for compositions and methods useful for preventing and treating autoimmune diabetes. The present invention satisfies this need.

### SUMMARY OF THE INVENTION

It is disclosed herein that nIgM therapy is useful in preventing the onset and progression of autoimmune diabetes, in treating autoimmune diabetes, and in promoting islet graft survival.

This application discloses the results of studies to: a) investigate whether purified, polyclonal mouse serum nIgM autoantibody therapy could have a functional role in inhibiting the onset and progression of T1D when administered in a NOD mouse model of autoimmune T1D; b) determine if intervention at the prehyperglycemic, late-stage of disease development or after the establishment of overt diabetes might be feasible; and c) investigate if administration of nIgM could prolong graft survival following syngeneic and allogeneic islet transplantation by mitigating inflammation.

The present application discloses, inter alia, the unexpected result that nIgM therapy inhibits onset and progression of autoimmune diabetes when initiated in the early- and late-onset stage of prehyperglycemic beta cell destruction. Also disclosed herein is that nIgM therapy may result in permanent protection from autoimmune beta cell destruction. The present application further discloses that nIgM therapy inhibits insulitis and promotes beta cell neogenesis. It is also disclosed herein that nIgM therapy with islet transplantation restores normoglycemia in overtly diabetic NOD mice and that nIgM therapy promotes syngeneic and allogeneic graft survival. Therefore, the present invention encompasses the use of nIgM therapy to enhance islet transplant survival in subjects receiving a transplant. It is also demonstrated herein that nIgM therapy delays the incidence/onset of diabetes in the adoptive transfer of disease model and demonstrates the induction of beta cell specific hyporesponsiveness or tolerance in an adoptive transfer of tolerance model. Further disclosed herein is that nIgM inhibits proinflammatory cytokine production from activated splenocytes from prediabetic NOD mice in vitro and it does not affect islet function.

The present invention provides, inter alia, for the use of nIgM therapy in preventing the onset, progression, and recurrence of autoimmune Type 1 Diabetes Mellitus, as well as treating Type 1 Diabetes Mellitus. Useful IgM of the invention can be found in, for example, blood, plasma, platelet-poor plasma, serum, B cells, and bone marrow. In one aspect, the IgM is human, but one of ordinary skill in the art will appreciate that IgM from other mammals can be used in some cases, including, primate, horse, and mouse. When all IgM's of the blood are used or purified together it is referred to as polyclonal IgM. In one aspect, the nIgM is purified. In one aspect, the serum is obtained from the subject who will be receiving the therapy. In addition, the present invention provides for the use of polyclonal serum nIgM therapy in islet transplantation, promoting longitudinal and functional graft survival, and preventing recurrence of the disease following islet transplantation. In one aspect, the therapy of the invention promotes islet allograft survival.

In one embodiment, the present invention provides a method of preventing or treating Type 1 Diabetes, said method comprising administering to a subject in need thereof a pharmaceutical composition comprising an effective amount of naturally occurring IgM (nIgM), a pharmaceutically-acceptable carrier, and optionally at least one additional therapeutic agent.

Depending on whether nIgM is being administered as a preventative or as a treatment of early or late stage diseases or disorders or in conjunction with transplantation, the amount of nIgM administered and the frequency of administration can vary. For example, in one embodiment, doses can range from about 0.01 mg of nIgM/kg body weight to about 30 mg nIgM/kg body weight. In one aspect, doses can range from about 0.1 mg nIgM/kg body weight to about 25 mg nIgM/kg body weight. In another aspect, doses can range from about 1.0 mg nIgM/kg body weight to about 20 mg nIgM/kg body weight. In another aspect, doses can range from about 3.0 mg nIgM/kg body weight to about 15 mg nIgM/kg body weight. In one aspect, doses of greater than about 10, 15, 20, or 50 mg nIgM/kg body weight are administered.

The nIgM of the invention can be administered multiple times per day or week or month. In one aspect, it is administered at least twice a month. In one aspect, it is administered at least twice a week. In one aspect, it is administered at least twice a day. The doses and regimen can be varied based on the age, sex, and health of the subject as well as on the disease, disorder, or condition being treated or prevented. In one aspect, a subject receives up to about 5 total doses of nIgM, in another aspect up to about 10 doses, and in yet another aspect up to about 50 doses. In one aspect, at least about 10, or 20, or 50, or 100 doses are administered. When more than one dose is administered, each dose does not have to be the same. For example, in one embodiment, a first dose can be larger than subsequent doses.

The treatments of the invention can be used in conjunction with transplantation of, for example, islets or pancreas.

One of ordinary skill in the art will appreciate that useful IgM can be obtained from different sources and can be polyclonal. In one embodiment, the nIgM is polyclonal serum IgM. In one aspect, it has been purified from serum. In one aspect, the serum is human serum. In one aspect, the serum has been obtained from the subject who is to undergo treatment. In one aspect, the source is a person with Type 1 Diabetes and in another it is from a person without Type 1 Diabetes. In one aspect, nIgM is purified by column chromatography and then concentrated. Other techniques, such as affinity chromatography or initial dialysis of serum before column chromatography can be used, but can lead to decreased biological activity of the nIgM. In one aspect, the purified nIgM is dialyzed into a pharmaceutical composition, buffer, or medium and filter sterilized. The invention further encompasses the use of biologically active fragments of nIgM.

In one embodiment, a pharmaceutical composition comprising an effective amount of nIgM is administered beginning in the early or late onset stage of prehyperglycemic beta cell destruction and inhibits beta cell destruction. In one aspect, the treatment inhibits insulitis and promotes beta cell neogenesis.

In one embodiment, a pharmaceutical composition of the invention comprising nIgM is administered by a method selected from intravenously, intraperitoneally, and intraarterially. In one aspect, it is administered locally.

In one aspect, at least one additional therapeutic agent is used. In one aspect, the at least one additional therapeutic agent is a cell. The cell can be selected from the group consisting of cord blood cells, islet cells, dendritic cells, regulatory T cells, stem cells, insulin-producing cells, mesenchymal stem cells, induced pluripotent stem cells, embryonic stem cells, hematopoietic stem cells, adipocyte stem cells, and neural stem cells.

In one aspect, a pharmaceutical composition comprising nIgM is administered to a subject before any signs of Type 1 Diabetes are present or shortly after the first signs. In one aspect, it is administered after the establishment of moderate Type 1 Diabetes. In another aspect, it is administered after the establishment of severe Type 1 Diabetes and can be administered in conjunction with islet transplantation for such a subject. In one aspect, it is administered in the early onset stage of prehyperglycemic beta cell destruction. In another aspect, it is administered in the late onset stage of prehyperglycemic beta cell destruction.

The present invention provides for administering nIgM to prevent or treat Type 1 Diabetes and has at least one of the following effects: inhibits insulitis; promotes beta cell neogenesis; induces beta cell specific hyporesponsiveness; inhibits insulin autoantibody production; inhibits beta cell destruction; inhibits periductular/perivascular inflammation in the pancreas.

In one embodiment the additional therapeutic agents are selected from the group consisting of anti-microbial agents, anti-inflammatory agents, anesthetic agents, analgesic agents, steroids, glucagon-like peptide 1 receptor agonists, dipeptidyl peptidase IV inhibitors, and immunodulatory agents.

In one embodiment, at least one additional therapeutic agent is administered and in another at least two are administered. When additional therapeutic agents are administered, in aspect, they are administered as part of the pharmaceutical composition comprising nIgM. In another aspect, they are administered in a separate pharmaceutical composition. In one aspect, the additional therapeutic agents are not administered each time the nIgM therapy is administered.

It is understood that additional therapeutic agents can include drugs, biologic molecules and cells.

In addition, IgM therapy is beneficial in prevention of autoimmune diabetes and treating islet transplantation subjects in combination with cellular therapy (e.g., cord blood, dendritic cell, regulatory T cells infusions etc) or stem cell therapy (e.g. generation of insulin-producing cells (IPCs) derived from pancreatic ductal/acinar tissue/islets OR IPCs derived from MSCs, induced pluripotent stem cells or embryonic, hematopoietic, cord blood, adipocyte, neural stem cell etc by stem cell technology). IgM therapy can be combined with therapies useful in preserving and expanding beta cell mass, for instance, GLP-1 receptor agonists like exendin-4 that stimulate beta cell proliferation and neogenesis and inhibit beta cell apoptosis, DPPIV inhibitors that increase beta cell insulin content.

In one embodiment, polyclonal serum IgM therapy is useful when administered in conjunction with islet transplantation, promoting longitudinal and functional graft survival and preventing recurrence of the disease following islet transplantation. This therapy can be done alone or in combination with antigen specific or non-specific immunosuppressive/immunomodulatory therapy, for instance Thymoglobulin (anti-thymocyte globulin, ATG) or Alemtuzumab (Campath-1H, monoclonal anti CD52 Ab) or hOKT3 gamma or Anti-CD25 induction) coupled with a sirolimus-based, prednisone-free maintenance regimen in combination with mycophenolate mofetil and low Tacrolimus/cyclosporine/FTY720/daclizumab, etc. This concept extends to other autoimmune or immune-associated diseases and organ/cellular transplantations.

In addition, the nIgM therapy of the present invention, alone or in combination, is useful in prevention of autoimmune diabetes and islet transplantation in combination with cellular therapy (e.g., cord blood, dendritic cell, regulatory T cells infusions etc) or stem cell therapy (e.g., generation of insulin-producing cells (IPCs) derived from pancreatic ductal/acinar tissue/islets OR IPCs derived from MSCs, induced pluripotent stem cells or embryonic, hematopoietic, cord blood, adipocyte, neural stem cell etc by stem cell technology). nIgM therapy is also useful in combination therapy when combined with therapies useful in preserving and expanding beta cell mass, for instance, GLP-1 receptor agonists like exendin-4 that stimulate beta cell proliferation and neogenesis and inhibit beta cell apoptosis, and DPPIV inhibitors that increase beta cell insulin content.

Thus, IgM therapy in combination with methods involving cellular/organ replacement and/or immune modulation or suppression and cellular neogenesis/regeneration initiated during various stages in the history of autoimmune diabetes (primary, secondary, or tertiary stage) or islet transplantation is encompassed by the present invention. The present invention further encompasses clinical applications that include, but are not limited to, administration of therapeutic doses of purified autologous polyclonal serum IgM to prediabetics to prevent onset or progression of disease, as well as administration to the islet transplant recipient pretransplant or in the induction phase before immune suppression to reduce the risk of rejection, promote graft survival, prevent recurrence of the disease, or development of a vaccine administered to patients before transplantation to increase in vivo production of nIgM-ALA. The commercial potential of providing nIgM similar to IgG (I.V.) administration for the protection against onset and progression of T1DM is tremendous as is the administration of nIgM prior to islet transplantation as a therapeutic intervention that promotes graft survival and recurrence of the disease.

Thus, nIgM therapy in combination with interventions and therapies involving cellular/organ replacement and/or immune modulation or suppression and cellular neogenesis/regeneration initiated during various stages in the history of autoimmune diabetes (primary, secondary, or tertiary stage) or islet transplantation is encompassed by the present invention. The concept encompasses other organ/cellular transplantations and autoimmune or immune-associated diseases. The present application further encompasses combination therapies comprising administering nIgM in combination with other drugs or compounds useful for treating or preventing the diseases and disorders described herein.

The nIgM therapy of the invention can be administered alone or performed as a combination therapy, including for example, antigen specific or non-specific immunosuppressive/immunomodulatory therapy (for instance Thymoglobulin {anti-thymocyte globulin, ATG} or Alemtuzumab {Campath-1H, monoclonal anti CD52 Ab} or hOKT3 gamma or Anti-CD25 induction) coupled with a sirolimus-based, prednisone-free maintenance regimen in combination with mycophenolate mofetil and low Tacrolimus/cyclosporine/FTY720/daclizumab, etc. This concept extends to other autoimmune or immune-associated diseases and organ/cellular transplantations.

In one aspect, the nIgM therapy reduces proinflammatory cytokines. In one aspect, the therapy causes an increase in Treg cells (formerly called T suppressors).

The present invention therefore provides compositions and methods useful for enhancing graft survival. In one embodiment, the invention provides a method of enhancing survival of an islet or pancreatic graft in a subject, comprising administering to a subject in need thereof a pharmaceutical composition comprising an effective amount of naturally occurring IgM (nIgM), a pharmaceutically-acceptable carrier, and optionally at least one additional therapeutic agent. In one aspect, the method restores normoglycemia in a Type 1 Diabetic. In one aspect, the method does not affect islet function. In one aspect, the method prevents recurrence of the disease and promotes beta cell specific hyporesponsiveness or tolerance. In one aspect, the method promotes beta cell neogenesis (regeneration).

One of ordinary skill in the art will appreciate that the therapy disclosed herein can also be used in combination with other drugs and therapeutic agents, including, but not limited to, antimicrobial agents such as antibiotics. The present therapy can be administered in a manner determined by a clinician to be suitable for the particular subject. Timing and dosage of the therapy can also be varied.

In one embodiment, nIgM is administered before a graft is transplanted. In another embodiment, nIgM is administered after a graft is transplanted. In another embodiment, nIgM is administered before and after a graft is transplanted. In one aspect, the nIgM is obtained from a source including, but not limited to, blood, plasma, platelet-poor plasma, or serum. In one aspect, the IgM is polyclonal serum nIgM. In one aspect, the serum has been purified. In one aspect, the nIgM has been purified. In one aspect, the purified nIgM has been purified by column chromatography and then concentrated. In one aspect, the purified nIgM has been dialyzed into a pharmaceutical composition, buffer, or medium and filter sterilized. In one aspect, the nIgM is obtained from the subject.

In one embodiment, the nIgM is administered to a transplant subject at a dose of about 0.01 mg nIgM/kg body weight to about 30 mg nIgM/kg body weight. In one aspect, the nIgM is administered at a dose of about 0.1 mg nIgM/kg body weight to about 25 mg nIgM/kg body weight. In one aspect, the nIgM is administered at a dose of about 1.0 mg nIgM/kg body weight to about 20 mg nIgM/kg body weight. In one aspect, the nIgM is administered at least twice. In one aspect, the pharmaceutical composition comprising nIgM is administered by a method selected from intravenously, intraperitoneally, and intraarterially. In one aspect, the pharmaceutical composition is administered up to about 5 times, in another the pharmaceutical composition is administered up to about 10 times, and in another the pharmaceutical composition is administered up to about 50 times. In one aspect, doses of greater than about 10, 15, 20, or 50 mg nIgM/kg body weight are administered.

In one embodiment, a transplant recipient receiving nIgM therapy of the invention, also further receives at least one additional therapeutic agent. In one aspect, the therapeutic agent is a cell. In one aspect, the cell is selected from the group consisting of cord blood cells, islet cells, dendritic cells, regulatory T cells, stem cells, insulin-producing cells, mesenchymal stem cells, induced pluripotent stem cells, embryonic stem cells, hematopoietic stem cells, adipocyte stem cells, and neural stem cells.

In one aspect, the treatment is effective in inhibiting periductular/perivascular inflammation in the pancreas.

In one embodiment, the additional therapeutic agent is selected from the group consisting of anti-microbial agents, anti-inflammatory agents, anesthetic agents, analgesic agents, steroids, glucagon-like peptide 1 receptor agonists, dipeptidyl peptidase IV inhibitors, and immunodulatory agents.

The present invention further provides kits. For example, the invention provides a kit for use in preventing or treating Type 1 Diabetes or for enhancing islet graft survival. In one aspect, the kit comprises at least one dose of nIgM, optionally at least one additional therapeutic agent, an applicator, and an instructional material for the use thereof.

Various aspects and embodiments of the invention are described in further detail below.

### BRIEF DESCRIPTION OF THE DRAWINGS

FIG. 1. nIgM therapy inhibits onset and progression of autoimmune diabetes. Groups of NOD mice were injected biweekly, beginning at 4 to 5 or 10 to 11 weeks of age with nIgM or PBS (A) or BSA or mouse IgG up to 18 weeks of age (B). Diabetes incidence was monitored by measuring tail vein BG levels and hyperglycemia defined as BG 250 mg/dL or more on 3 successive days. The arrows indicate IgM therapy initiated when mice are 4-5 weeks of age or initiated when mice are 9-11 or 10-11 weeks of age.

FIG. 2. nIgM therapy inhibits insulitis and promotes beta cell neogenesis. H&E stained pancreas sections were examined histologically. Representative sections are shown. 2A. Islet from non-diabetic 4-5 week old NOD mouse; 2B. Islet from 18-week-old diabetic NOD mouse showing insulitis and, 2C. perivascular and periductular inflammation; 2D. Islet from 18 weeks old nIgM-treated, non-diabetic NOD mouse showing no insulitis and mild perivascular and periductular inflammation; and 2E. Islet from 18 weeks old, nIgM-treated non diabetic NOD mouse showing mild insulitis/peri-insulitis and beta cell neo-genesis. (Thick arrow indicates islets. Thin arrow indicates insulitis, peri-insulitis, or periductular/perivascular inflammation).

FIG. 3. nIgM therapy promotes syngeneic graft survival. 50 C57Bl6 syngeneic islets were transplanted under the renal capsule of Streptozotocin-induced diabetic C57Bl/6 mice receiving nIgM or PBS/BSA (75 ug triweekly). Mice with BG levels≧300 mg/dL were considered diabetic. nIgM therapy reduced the time taken to return to normoglycemia, significantly improving islet graft survival compared to the control group (p<0.001).

FIG. 4. nIgM therapy promotes allogeneic graft survival. 300 allogeneic BALB/c islets were transplanted under the renal capsule of STZ induced, diabetic C57Bl/6 mice receiving nIgM or PBS/BSA (75 ug triweekly). A third group of control diabetic mice received nIgM therapy alone without islet transplants. BG levels≧300 mg/dL was an indication of graft rejection. nIgM therapy significantly increased the mean survival time of islet allografts compared to controls (p<0.001).

FIG. 5. nIgM delays the incidence/onset of diabetes (appearance of hyperglycemia) in adoptive transfer model. nIgM therapy significantly delays the incidence/onset of diabetes (appearance of hyperglycemia) following adoptive transfer of diabetic NOD splenocytes (FIG. 5A) or CD3 T cells (FIG. 5B) into NOD scid recipient mice when compared to control mice receiving BSA (p<0.001). Transfer of splenocytes or CD3 T cells from nondiabetic 2-3 week old NOD mice did not transfer disease.

FIG. 6. nIgM inhibits proinflammatory cytokine production from activated splenocytes in vitro. nIgM significantly inhibits proinflammatory cytokine production in vitro from splenocytes obtained from 3-6 week old NOD mice (n=4) that were activated with LPS and soluble anti-CD3 and cultured for 4 days at 37° C., 5% CO₂, with BSA or purified nIgM.

FIG. 7. IgM therapy induces beta cell specific hyporesponsiveness (tolerance). The ordinate represent time to hyperglycemia or diabetic incidence in days and the abscissa represents the groups (4).

FIG. 8. IgM therapy inhibits insulin auto-antibody production. This figure graphically demonstrates that IgM therapy inhibits insulin auto-antibody production in the NOD mouse model of diabetes. The ordinate represents amount of mIAA: Index=(sample Δ cpm−negative control Δ cpm)/(positive control Δ cpm−negative control Δ cpm). The abscissa represents the seven groups.

### DETAILED DESCRIPTION

### Abbreviations and Acronyms—

Ag—antigen
ALS—anti-lymphocyte serum
ATG—anti-thymocyte globulin
ASC—adipose tissue stem cell
b.w.—body weight
BG—blood glucose
CB-SC—cord blood stem cells
cmf—calcium and magnesium free
DPPIV—dipeptidyl peptidase IV, also referred to as DPP-4
GADA—GAD65 autoantibody
GLP-1 receptor—glucagon-like peptide 1 receptor
IAA—insulin autoantibodies
ICA—islet cell autoantibodies
IFN—interferon
IgM—immunoglobulin M
IgM-ALA—IgM antileukocyte autoantibodies
IL—interleukin
iNKT—invariant natural killer T cells
IPCs—insulin-producing cells
mIAA—micro IAA assay
MSC—mesenchymal stem cell
nIgM—natural or naturally occurring IgM
nIgM-ALA—naturally occurring or natural IgM-ALA
NOD—non-obese diabetic
PBS—phosphate buffered saline
scid—severe combined immunodeficiency
STZ—streptozotocin
T1D—type 1 diabetes
TNF—tumor necrosis factor
Treg—regulatory T cells

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound or cell for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds may also be used to treat symptoms associated with the injury, disease or disorder, including, but not limited to, pain and inflammation. Such compounds or agents include, but are not limited to drugs, antimicrobials, growth factors, cytokines, etc.

As use herein, the terms “administration of” and or “administering” a compound should be understood to mean providing a compound of the invention or a prodrug of a compound of the invention to a subject in need of treatment.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

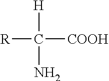

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

The term “autologous” as used herein refers to a situation in which the donor and recipient are the same person.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, skin, hair, tissue, blood, plasma, cells, sweat and urine.

The term “biweekly” means twice per week.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds or proteins, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Graft” refers to any free (unattached) cell, tissue, or organ for transplantation.

“Allograft” refers to a transplanted cell, tissue, or organ derived from a different animal of the same species.

“Xenograft” refers to a transplanted cell, tissue, or organ derived from an animal of a different species.

The term “IgM” can be used, depending on the context, to include natural, synthetic, or recombinant IgM as well as IgM from various sources, including polyclonal serum IgM and purified naturally occurring IgM.

By the term “immunizing a human against an antigen” is meant administering to the human a composition, a protein complex, a DNA encoding a protein complex, an antibody or a DNA encoding an antibody, which elicits an immune response in the human which immune response provides protection to the human against a disease caused by the antigen or an organism which expresses the antigen.

“Inappropriate apoptosis” of cells refers to apoptosis (i.e. programmed cell death) which occurs in cells of an animal at a rate different from the range of normal rates of apoptosis in cells of the same type in an animal of the same type which is not afflicted with a disease or disorder.

As used herein, the term “induction of apoptosis” means a process by which a cell is affected in such a way that it begins the process of programmed cell death, which is characterized by the fragmentation of the cell into membrane-bound particles that are subsequently eliminated by the process of phagocytosis.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, “islet” refers to pancreatic islet.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with a pathogenic agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease. This can be equated with “preventing”.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides.

The term “peptide” typically refers to short polypeptides.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

A “receptor” is a compound that specifically binds to a ligand.

A “ligand” is a compound that specifically binds to a target receptor.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

The term “substantially pure” describes a compound, e.g., a protein or polypeptide that has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

The term “triweekly” means three times per week.

By the term “vaccine,” as used herein, is meant a composition which when inoculated into an animal has the effect of stimulating an immune response in the animal, which serves to fully or partially protect the animal against a disease or its symptoms. The term vaccine encompasses prophylactic as well as therapeutic vaccines. A combination vaccine is one which combines two or more vaccines.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### EMBODIMENTS

In one embodiment, the serum is autologous.

In one embodiment, the invention encompasses peptides, proteins, and fragments, homologs, derivatives, and modifications thereof. The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

It will be appreciated, of course, that the peptides may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.
   Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In one embodiment, the inhibitor comprises from about 0.0001% to about 15% by weight of the pharmaceutical composition.

The invention should be construed to include various methods of administration, including intravenous, intraperitoneal, topical, oral, intramuscular, intrathecal, vaginal, rectal, subcutaneous, and buccal. The route(s) of administration will be readily apparent to the skilled artisan and will depend upon any number of factors including the type and severity of the disease being treated, the type and age of the veterinary or human subject being treated, and the like.

The invention relates to the administration of an identified compound (antibody, other therapeutic agent, etc.) in a pharmaceutical composition to practice the methods of the invention, the composition comprising the compound or an appropriate analog, homolog, derivative, modification, or fragment of the compound and a pharmaceutically-acceptable carrier.

The present invention also encompasses pharmaceutical and therapeutic compositions comprising the compounds of the present invention.

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

Suitable preparations of vaccines include injectables, either as liquid solutions or suspensions, however, solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

The invention is also directed to methods of administering the compounds of the invention to a subject. In one embodiment, the invention provides a method of treating a subject by administering compounds identified using the methods of the invention description. Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating and vaccinating a subject in need of such treatment is provided. The method comprises administering a pharmaceutical composition comprising at least one compound of the present invention to a subject in need thereof. Compounds identified by the methods of the invention can be administered with known compounds or other medications as well.

The invention also encompasses the use of pharmaceutical compositions of an appropriate compound, and homologs, fragments, analogs, or derivatives thereof to practice the methods of the invention, the composition comprising at least one appropriate compound, and homolog, fragment, analog, or derivative thereof and a pharmaceutically-acceptable carrier.

In one embodiment, the pharmaceutical compositions useful for practicing the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 100 g/kg/day.

Pharmaceutically acceptable carriers which are useful include, but are not limited to, glycerol, water, saline, ethanol, and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

For in vivo applications, the peptides of the present invention may comprise a pharmaceutically acceptable salt. Suitable acids which are capable of forming such salts with the compounds of the present invention include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g. 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the present invention can be prepared in a manner fully within the skill of the art.

The peptides of the invention, pharmaceutically acceptable salts thereof, or pharmaceutical compositions comprising these compounds may be administered so that the compounds may have a physiological effect. Administration may occur enterally or parenterally; for example orally, rectally, intracisternally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g. intravenous bolus injection, intravenous infusion, intra-arterial bolus injection, intra-arterial infusion and catheter instillation into the vasculature), peri- and intra-target tissue injection (e.g. peri-tumoral and intra-tumoral injection), subcutaneous injection or deposition including subcutaneous infusion (such as by osmotic pumps), intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Controlled- or sustained-release formulations of a pharmaceutical composition of the invention may be made using conventional technology.

The source of active compound to be formulated will generally depend upon the particular form of the compound. Small organic molecules and peptidyl or oligo fragments can be chemically synthesized and provided in a pure form suitable for pharmaceutical/cosmetic usage. Products of natural extracts can be purified according to techniques known in the art. Recombinant sources of compounds are also available to those of ordinary skill in the art.

Liquid derivatives and natural extracts made directly from biological sources may be employed in the compositions of this invention in a concentration (w/v) from about 1 to about 99%. Fractions of natural extracts and protease inhibitors may have a different preferred rage, from about 0.01% to about 20% and, more preferably, from about 1% to about 10% of the composition. Of course, mixtures of the active agents of this invention may be combined and used together in the same formulation, or in serial applications of different formulations.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention that may be administered to an animal, preferably a human, will vary depending upon any number of factors, including but not limited to, the type of animal and type of disease state being treated, the age of the animal, health of the animal, and the route of administration.

The compound (antibodies, etc.) can be administered to a subject as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the subject, etc.

The compounds (antibodies, etc.) of the invention may be administered to, for example, a cell, a tissue, or a subject by any of several methods described herein and by others which are known to those of skill in the art.

When used in vivo for therapy, the antibodies of the subject invention are administered to the patient in therapeutically effective amounts (i.e., amounts that have desired therapeutic effect). They can be administered parenterally. The dose and dosage regimen will depend upon the degree of the disease, disorder or condition, the characteristics of the particular antibody used, e.g., its therapeutic index, the patient, and the patient's history. Optionally, the administration is made during the course of adjunct therapy such as antimicrobial treatment, or administration of tumor necrosis factor, interferon, or other cytoprotective or immunomodulatory agent.

For parenteral administration, the antibodies can be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

There is evidence that complement activation in vivo leads to a variety of biological effects, including the induction of an inflammatory response and the activation of macrophages (Unanue and Benecerraf, Textbook of Immunology, 2nd Edition, Williams & Wilkins, p. 218 (1984)). The increased vasodilation accompanying inflammation can increase the ability of various agents to localize in infected cells. Therefore, antigen-antibody combinations of the type specified by this invention can be used therapeutically in many ways. Additionally, purified antigens (Hakomori, Ann. Rev. Immunol. 2:103, 1984) or anti-idiotypic antibodies (Nepom et al., Proc. Natl. Acad. Sci. USA 81: 2864, 1985; Koprowski et al., Proc. Natl. Acad. Sci. USA 81: 216, 1984) relating to such antigens could be used to induce an active immune response in human patients. Such a response includes the formation of antibodies capable of activating human complement and mediating ADCC and by such mechanisms cause infected cell destruction.

Optionally, the antibodies of this invention are useful as antibody-cytotoxin conjugate molecules, as exemplified by the administration for treatment of neoplastic disease.

The antibody compositions used in therapy are formulated and dosages established in a fashion consistent with good medical practice taking into account the disorder to be treated, the condition of the individual patient, the site of delivery of the composition, the method of administration, and other factors known to practitioners. The antibody compositions are prepared for administration according to the description of preparation of polypeptides for administration, infra.

As is well understood in the art, biospecific capture reagents include antibodies, binding fragments of antibodies which bind to activated integrin receptors on metastatic cells (e.g., single chain antibodies, Fab′ fragments, F(ab)′₂ fragments, and scFv proteins and affibodies (Affibody, Teknikringen 30, floor 6, Box 700 04, Stockholm SE-10044, Sweden; See U.S. Pat. No. 5,831,012, incorporated herein by reference in its entirety and for all purposes)). Depending on intended use, they also can include receptors and other proteins that specifically bind another biomolecule.

Hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Application No. 20030022244.

Initially, a predetermined target object is chosen to which an antibody can be raised. Techniques for generating monoclonal antibodies directed to target objects are well known to those skilled in the art. Examples of such techniques include, but are not limited to, those involving display libraries, xeno or humab mice, hybridomas, and the like. Target objects include any substance which is capable of exhibiting antigenicity and are usually proteins or protein polysaccharides. Examples include receptors, enzymes, hormones, growth factors, peptides and the like. It should be understood that not only are naturally occurring antibodies suitable for use in accordance with the present disclosure, but engineered antibodies and antibody fragments which are directed to a predetermined object are also suitable.

The composition may further comprise an effective amount of at least one additional therapeutic agents which may be useful for the type of injury, disease, or disorder being treated. Additional therapeutic agents include, but are not limited to, anesthetic, analgesic, antimicrobial, steroid, growth factor, cytokine, and anti-inflammatory agents.

In another aspect, the agent is at least one analgesic. In one aspect, the agent is at least one anesthetic. In yet another aspect, the agent is an additional therapeutic drug.

In a further aspect, the additional therapeutic agent is an antimicrobial agent. In one aspect, the antimicrobial agent is an antibacterial agent. In another aspect, the antimicrobial agent is an antifungal agent. In yet another aspect, the antimicrobial agent is an antiviral agent.

In another aspect, the agent is selected from aspirin, pentoxifylline, and clopidogrel bisulfate, or other angiogenic, or a rheologic active agent.

A list of the types of drugs, and specific drugs within categories which are encompassed within the invention is provided below and are intended be non-limiting examples.

Antimicrobial agents include: silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, antimicrobial peptides (e.g., natural and synthetic origin), Neosporin (i.e., Bacitracin, Polymyxin B, and Neomycin), Polysporin (i.e., Bacitracin and Polymyxin B). Additional antimicrobials include topical antimicrobials (i.e., antiseptics), examples of which include silver salts, iodine, benzalkonium chloride, alcohol, hydrogen peroxide, and chlorhexidine.

Anesthetics: Useful anesthetic agents include benzocaine, lidocaine, bupivocaine, dibucaine, mepivocaine, etidocaine, tetracaine, butanilicaine, and trimecaine.

Analgesic: Acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin.

Anti-inflammatory: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Antinauseant: Buclizine Hydrochloride; Cyclizine Lactate; Naboctate Hydrochloride.

Antineutropenic: Filgrastim; Lenograstim; Molgramostim; Regramostim; Sargramostim.

One type of administration encompassed by the methods of the invention is parenteral administration, which includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, and intrasternal injection, and kidney dialytic infusion techniques.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, and intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

Additional ingredients may be added to the pharmaceutical composition. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The compounds or nIgMs of the invention may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type, and age of the animal, etc.

The invention also includes a kit comprising the compounds of the invention and an instructional material that describes administration of the compounds. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression that can be used to communicate the usefulness of the compounds of the invention in the kit for effecting alleviation of the various diseases or disorders recited herein. Optionally, or alternately, the instructional material may describe one or more methods of alleviating the diseases or disorders. The instructional material of the kit of the invention may, for example, be affixed to a container that contains a compound of the invention or be shipped together with a container that contains the compounds. Alternatively, the instructional material may be shipped separately from the container with the intention that the instructional material and the compound be used cooperatively by the recipient.

The present invention further includes the use of adjuvants and additional therapeutic agents as part of the peptide treatment. For example, an additional therapeutic agent can include one of the other immunomodulating agents discussed herein, or an antimicrobial agent such as an antimicrobial or an antiviral.

Kits

The present invention should be construed to include kits for treating, preventing, or inhibiting diseases and disorders as described herein, including Type 1 Diabetes, and for enhancing islet graft survival. The invention further includes a kit comprising at least one IgM antibody of the invention, or a source of naturally occurring IgM, or biologically active derivatives, fragments, or homologs, thereof, a standard, and an instructional material which describes purifying or administering a compound of the invention to a cell or an animal, as well as optionally additional therapeutic agents. This should be construed to include other embodiments of kits that are known to those skilled in the art, such as a kit comprising a standard and a (preferably sterile) solvent suitable for dissolving or suspending the composition of the invention prior to administering the compound to a cell or an animal. Preferably the animal is a mammal More preferably, the mammal is a human.

In accordance with the present invention, as described above or as discussed in the Examples below, there can be employed conventional clinical, chemical, cellular, histochemical, biochemical, molecular biology, microbiology and recombinant DNA techniques which are known to those of skill in the art. Such techniques are explained fully in the literature. See for example, Sambrook et al., 1989 Molecular Cloning—a Laboratory Manual, Cold Spring Harbor Press; Glover, (1985) DNA Cloning: a Practical Approach; Gait, (1984) Oligonucleotide Synthesis; Harlow et al., 1988 Antibodies—a Laboratory Manual, Cold Spring Harbor Press; Roe et al., 1996 DNA Isolation and Sequencing: Essential Techniques, John Wiley; and Ausubel et al., 1995 Current Protocols in Molecular Biology, Greene Publishing.

The invention is now described with reference to the following Examples. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### EXAMPLES

### Example 1—

### Methods

nIgM Purification from Mouse Sera:

nIgM was purified by size-exclusion column chromatography (Sephacryl S-300 HR, GE Healthcare, Piscataway, N.J.) from irradiated, heat-inactivated (56° C.×1 h) WT-057Bl/6 murine sera (Innovative Research, Novi, Mich.), using previously described procedures [7] and with modifications detailed below. nIgM was not isolated by dialyzing sera in water or by ammonium chloride precipitation, since these techniques yield IgM with impaired functional activity. Column-purified nIgM was repassaged through Sephacryl S-300 to remove contaminating IgG and other proteins. With this approach, >92% of the protein fraction contained nIgM with <1% IgG, <3% albumin, and <1% other protein contaminants as determined by protein electrophoresis and ELISA techniques. We did not affinity purify nIgM Abs as such procedures (binding of nIgM to mannan binding protein or binding of nIgM to agarose coupled with goat anti-IgM antibodies) yield 10-15% of the starting IgM and have the potential of depleting certain IgM subsets. Purified nIgM was concentrated to 1.3-1.5 mg/ml (higher concentration lends to nIgM aggregation and precipitation), dialyzed against RPMI 1640, and millipored using a 0.45 μm filter, prior to use in cultures and for in vivo use. Purified IgM were stored at 4° C. to prevent precipitation that occurs when frozen.

Mice:

Female non-obese diabetic (NOD) mice littermates and NOD scid (severe combined immunodeficiency) mice were purchased from Jackson laboratories (Bar Harbor, Me.), whilst C57BL/6 and BALB/c mice used in islet transplantation and in vitro experiments were purchased from Charles River Laboratories (Wilmington, Mass.). The animals were housed in institutional vivaria. All procedures were approved by the Animal Care and Use Committee (ACUC) of University of Virginia and performed in accordance with National Institutes of Health and Institutional Animal Care and Use Guidelines.

nIgM Treatment Regimen in NOD Mice:

NOD mice were divided into 5 groups: two test groups consisting of female NOD littermates that received nIgM therapy beginning at 4-5 weeks of age (test group #1, n=33) or at 10-11 weeks of age (test group #2, n=20) and three control groups that received sterile phosphate buffered saline (PBS) (n=30), bovine serum albumin (BSA) (n=10) and mouse IgG (n=10) at the same time and in the case of BSA or IgG, in concentrations similar to nIgM administration. Briefly, mice received an initial intra-peritoneal injection of 100 ug purified nIgM, followed by 50-60 ug in 150 ul PBS biweekly until mice were 18 weeks old. These experiments were done using batches of 10 littermates in each group and repeated for statistical significance. NOD mice were deemed diabetic when tail vein blood glucose (BG) was ≧250 mg/dL (Accu-Chek Aviva glucose monitor) on three successive days. nIgM therapy was withdrawn after mice reached 18 weeks of age and were monitored for the appearance of T1D. In another experiment moderately diabetic mice (BG 250-340 mg/dL) received initial intra-peritoneal injection of 100 ug purified nIgM, followed by 75 ug triweekly, while severely diabetic mice (BG>350 mg/dL) received this dose along with minimal dose islet transplant (75 islets).

Islet Isolation and Transplantation:

10-16 weeks old female BALB/c served as donors in islet allo-transplantation experiments, whilst retired female C57Bl/6 mice were used as donors in syngeneic islet transplantation experiments. In some experiments, diabetic NOD mice recipients were transplanted with marginal dose NOD scid islets (75 islets). Briefly, islets were isolated by collagenase P digestion (Roche Molecular Biochemicals, Indianapolis, Ind.) and purified by density gradient centrifugation using Histopaque 1077 (Sigma-Aldrich, St. Louis, Mo.) according to a previously described procedure [15]. Following overnight culture (37° C. and 5% CO2), 300 BALB/c or 50 C57Bl/6 islets were transplanted under the kidney capsule of 10-12 week old female C57Bl/6 recipients that were rendered diabetic by a single IP injection of Streptozotocin (STZ, 250 mg/kg; Sigma) as described elsewhere [15].

Postoperatively, mice were administered analgesia and maintained at 35-37° C. prior to returning to the vivarium. Test group islet recipients received 100 ug nIgM predialyzed in RPMI 1640 (BioWhittaker, Walkersville, Md.) intraperitoneal, 24 hrs before transplantation and 75 ug triweekly every 48 hrs; whilst control group islet recipients received PBS/BSA. A third group did not receive islet transplant, but received nIgM therapy alone. Tail vein BG≧300 mg/dL on three successive days was indicative of graft rejection. In syngeneic islet transplantation, cure was defined as a return to normoglycemic state.

Adoptive Transfer of T1D:

5-6 week old immune-deficient NOD scid mice served as adoptive transfer recipients and were divided into two groups: nIgM therapy-receiving test group and BSA-injected control group. Recipients received 75 ug nIgM or BSA biweekly. 2×10⁷ splenocytes from diabetic NOD mice in 200 uls of sterile PBS were injected intraperitoneally into both groups and mice were monitored for hyperglycemia. Alternatively, CD3 T cell subsets were prepared by negative selection with magnetic beads using MagCellect Mouse CD3+ T Cell Isolation Kit (R&D Systems, Minneapolis, Minn.) according to the manufacturer's protocol. ˜97.5% purity was confirmed by flow cytometric analysis. 7.5×10⁶ CD3 T cells were transferred intraperitoneal into NOD scid recipients. In addition, a third group received either splenocytes or CD3 T cells from 2-3 week old prediabetic NOD mice, followed with BSA administration.

Histology:

Pancreases from NOD mice were excised, fixed (10% neutral buffered formalin), and prepared for routine paraffin histology. 5 um sections obtained at multiple levels were stained by hematoxylin and eosin (H&E) and observed for peri-insulitis, insulitis, perivascular and periductular inflammation, beta-cell regeneration etc.

Anti-CD3 and LPS Activation of Splenocytes:

Single cell splenocyte suspensions were obtained from prediabetic 2-5 week old NOD mice (n=4). Erythrocytes were removed using RBC lysing buffer (eBioscience, San Diego, Calif.). The cells were washed thrice with RPMI 1640 supplemented 2% fetal bovine serum (FBS) (Fisher Scientific, Pittsburgh, Pa.). Cell concentration was adjusted to 1×10⁶ cells/0.5 ml in RPMI 1640 containing 4.5 g/LD-Glucose, 2.383 g/L HEPES, L-glutamine, 110 mg/L sodium pyruvate, 1.5 g/L sodium bicarbonate and supplemented with 10% FBS, 1% Penicillin/streptomycin and beta-mercaptoethanol (1:1000) (Invitrogen, New York). The cells were initially activated with anti-mouse CD3e antibody (1 ug/ml; eBioscience 145-2C11) and lipopolysaccharide (0.35 ug/ml; Sigma-Aldrich) and cultured for 4 days at 37° C. in 5% CO2 in triplicate with or without IgM (˜3-10 ug). TNF-alpha, IFN-gamma and IL-17A in culture supernatants were quantitated using ELISA kits.

Statistical Analyses:

% diabetes incidence data was analyzed using Kaplan-Meier statistics, with significance determined by log-rank test was used to analyze the diabetes incidence data. Differences were considered statistically significant at p<0.001.

### Results

nIgM Therapy Inhibits Onset and Progression of Autoimmune Diabetes when Initiated in the Early and Late Onset Stage of Prehyperglycemic Beta Cell Destruction.

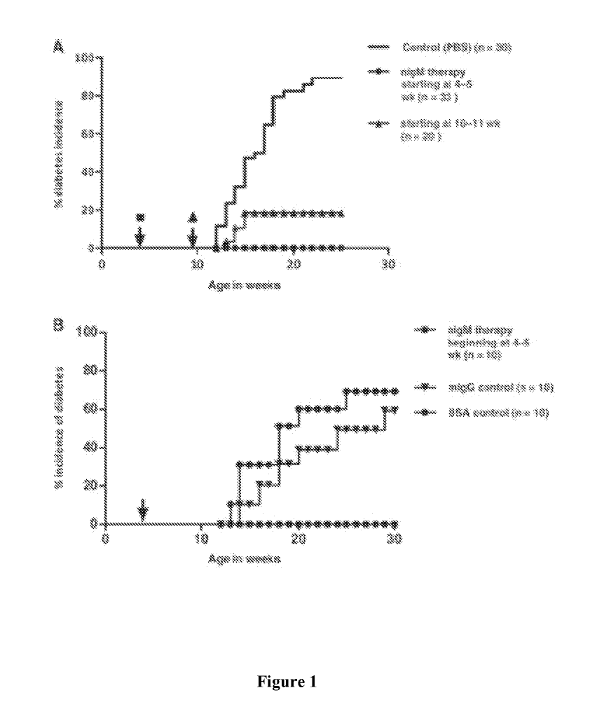

In our studies, 80% of the control mice (n=30) receiving saline became diabetic by 18-20 weeks of age and 90% by 25 weeks (FIG. 1A). BSA resulted in 70% diabetes (n=10) whilst IgG administration resulted in 50% (n=10) at 25 weeks of age (FIG. 1B). In contrast, none of nIgM-treated mice (0/30) that received therapy beginning at 4-5 weeks of age became diabetic (p<0.0001). Comparison of % diabetes incidence curves showed statistically significant difference between nIgM treated group compared to PBS, BSA and IgG (p<0.0001) (FIGS. 1A and B). These results indicate that nIgM therapy initiated in the early onset, prehyperglycemic phase of diabetes is capable of completely preventing the onset of autoimmune T1D development.

nIgM therapy initiated at 11 weeks of age, in the prehyperglycemic, late-onset phase of disease development, resulted in hyperglycemia in only 20% of nIgM-treated animals (n=20) at 25 weeks compared to 90% of controls (p<0.0001) (FIG. 1A). This is a clinically significant result since in the majority of studies, treatment begun after 9 weeks of age has proven ineffectual in protecting from beta cell destruction[16-17]. Thus, our results indicate that increasing serum levels of naturally-occurring IgM prevents the onset and progression of autoimmune diabetes.

nIgM Therapy May Result in Permanent Protection from Autoimmune Beta Cell Destruction.

In mice that received nIgM therapy beginning at 4-5 weeks of age, discontinuation of therapy at 18 week of age resulted in hyperglycemia in only 9/33 mice (˜27%) at 22-28 weeks post-discontinuation, indicating that nIgM therapy acts in an immunomodulatory capacity that results in possible permanent protection from beta cell destruction in the remaining ˜73% mice.

nIgM Therapy Inhibits Insulitis and Promotes Beta-Cell Neogenesis.

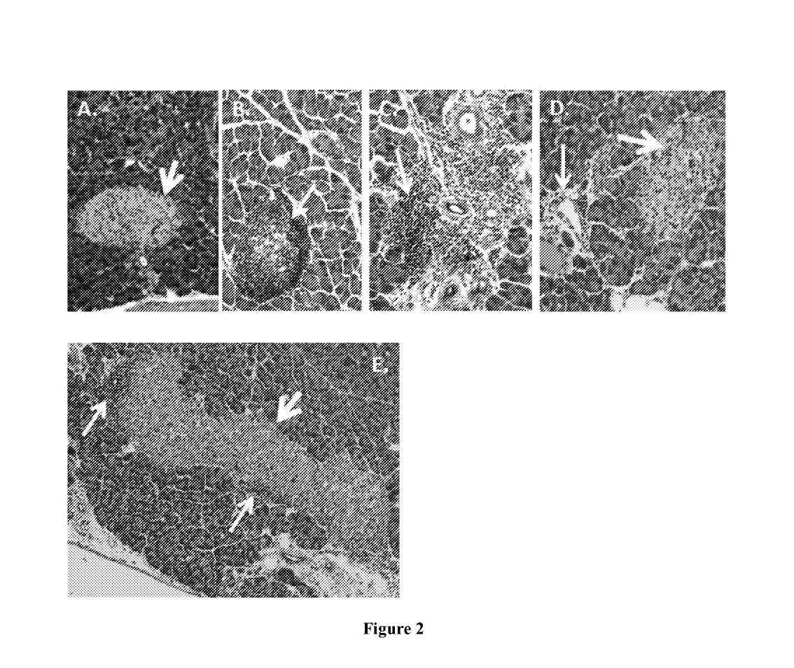

Examination of H&E-stained pancreas sections indicated the presence of severe insulitis as well as severe periductular and perivascular inflammation in the control diabetic mice group (FIG. 2B-C). In contrast, in pancreas sections obtained from the nIgM treated, non-diabetic mice group at 18-25 weeks of age, normal appearance or mild insulitis/peri-insulitis was observed in the nIgM-treated islets, accompanied by some periductular and perivascular inflammation (FIG. 2D). Additionally, numerous big islets in close proximity to ducts, a process classically observed in beta cell neogenesis, was also observed (FIG. 2E). These results indicate that nIgM therapy promotes beta cell regeneration while at the same time inhibits beta cell destruction.

nIgM Therapy with Islet Transplantation Restores Normoglycemia in Overtly Diabetic NODs.

In our studies, injecting control diabetic NOD mice that were mildly diabetic (BG 250-340 mg/dL) with 75 ug nIgM thrice weekly restored normoglycemia (n=5). This dose did not restore normoglycemia in severely diabetic mice (BG>350 mg/dL), (n=4). However, combining nIgM therapy with minimal dose islet transplant (75 islets) was able to permanently restore normoglycemia (n=4) in severely diabetic mice. This result is clinically significant since it demonstrates the therapeutic effect of elevated serum nIgM levels even after the initial appearance of mild hyperglycemia and indicates its clinical potential in islet transplantation.

nIgM Therapy Promotes Syngeneic and Allogeneic Graft Survival.

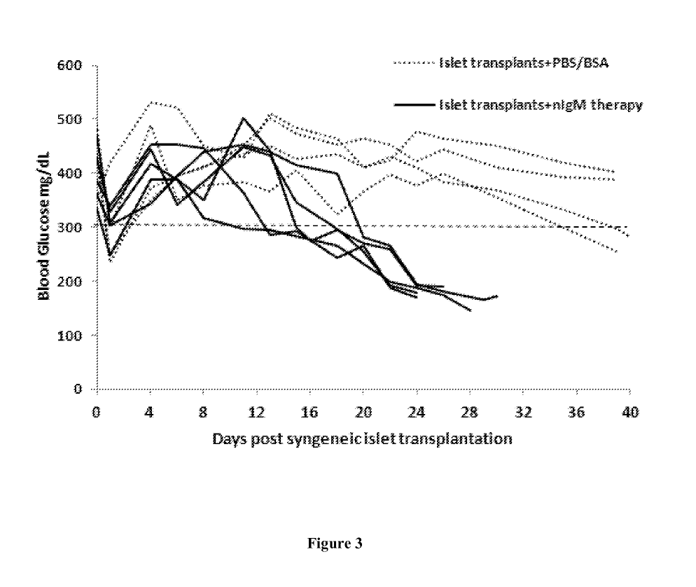

In the syngeneic islet transplantation studies using minimal dose islet transplant (50 islets), diabetic mice receiving 75 ug nIgM triweekly returned to normoglycemia within 15.4±3.6 days (n=5). Of 4 diabetic recipient mice that received syngeneic islet transplants with PBS/BSA instead of nIgM therapy, 2 mice returned to normoglycemia in 37±2.8 days while 2 did not achieve normoglycemia, remaining hyperglycemic (FIG. 3, p<0.001).

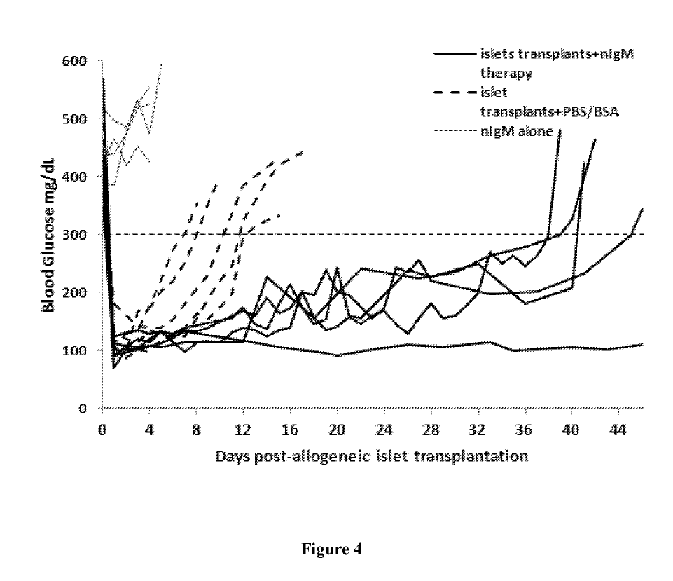

Similarly, in islet allotransplantation studies, of 5 diabetic C57Bl6 recipient mice that received 300 BALB/C islet transplants, the mean survival time was 41.2±3.3 days for 4 of the nIgM-treated recipients, whilst the fifth recipient remained permanently normoglycemic. In the control group that received PBS/BSA instead of nIgM therapy, the mean survival time of BALB/C islet allografts was 10.2±2.6 days for controls (n=5) (FIG. 4, p<0.001). These results demonstrate that nIgM therapy promotes longitudinal islet allograft survival and may prove to be an important adjunctive therapy to advance successful islet engraftment and transplant outcomes.

nIgM Therapy Delays the Incidence of Diabetes (Appearance of Hyperglycemia) in Adoptive Transfer Model.

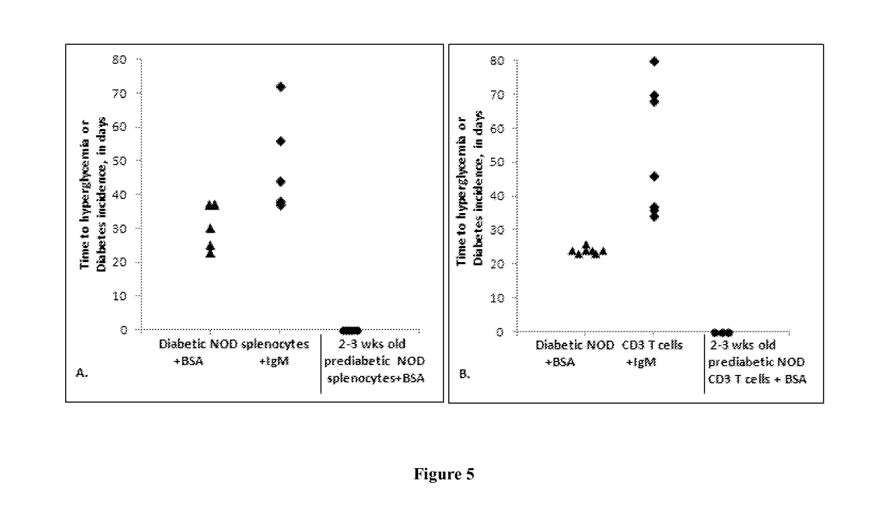

T1D can be adoptively transferred to immune-deficient NOD scid mice by splenocytes or CD3 T cells from diabetic NOD mice [18]. In our studies involving intraperitoneal adoptive transfer of splenocytes from diabetic NOD mice into 5-6 weeks old NOD scid recipient mice, the appearance of diabetes (hyperglycemia) was significantly delayed in recipient mice receiving nIgM therapy (49.4±14.7 days; n=5) when compared to the control group receiving PBS/BSA (30.4±6.5 days; n=5) (p<0.001) (FIG. 5A). A third group of recipient NOD scid mice that received splenocytes from 2-3 weeks old non-diabetic NOD mice (n=5) with PBS/BSA administration did not become diabetic (FIG. 5A).

Similar beneficial results were observed with nIgM therapy when CD3 T cells from diabetic NOD mice were transferred intraperitoneally to 5-6 week old NOD scid recipients. The appearance of diabetes was significantly delayed in NOD scid mice receiving nIgM therapy (53.0±19.1 days; n=7) when compared to the control group (24.0±1.0 days; n=7) that received PBS/BSA (p<0.001) (FIG. 5B). Recipient NOD scid mice that received CD3 T cells from 2-3 weeks old non-diabetic NOD mice (n=3) with PBS/BSA administration did not become diabetic (FIG. 5B). These results indicate that elevation of serum nIgM levels delays the incidence of diabetes through immunomodulation of T cells, suggesting an immunoregulatory role in the possible development of tolerance.

nIgM Inhibits Proinflammatory Cytokine Production from Activated Splenocytes from Prediabetic NOD Mice In Vitro.

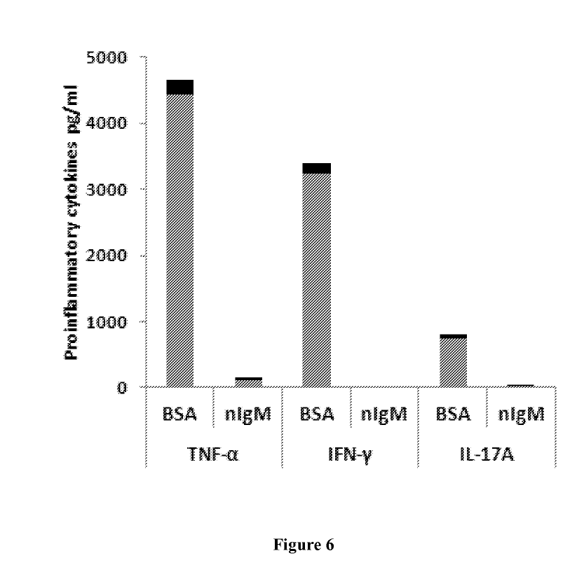

T1D is thought to be a Th1-dominance disease with type 1 cytokines such as IFN-gamma, TNF-alpha, IL-2, IL-12, and IL-18 actively involved in β-cell destruction [4]. Of interest is the recent discovery that Th17 cells are potent inducers of tissue inflammation and autoimmunity and may have a role in T1D [19-21]. Therefore, we decided to study the effect of nIgM on in vitro production of proinflammatory cytokine from activated 5×10⁶ cells freshly isolated splenocytes obtained from 3-5 week old NOD mice (n=4) and activated with LPS and soluble anti-CD3 were cultured for 4 days at 37° C., 5% CO2, without or with purified nIgM. Controls received BSA instead of nIgM. TNF-alpha, IFN-gamma, and IL-17A in supernatants were quantitated using ELISA kits. We found that nIgM significantly inhibited TNF-alpha secretion (activated splenocytes cultured with BSA vs. with nIgM; 4443.1±210.5 vs.101.1±52.5 pg/ml; p<0.001); IFN-gamma secretion (3244.2±160.6 vs. 0 pg/ml; p<0.0001) and IL-17A (732.2±66.4 vs. 5.3±9.2 pg/ml, p<0.0001) from LPS and anti-CD3 activated splenocytes in vitro when compared to controls (FIG. 6). These results indicate that nIgM acts by inhibiting the non specific, proinflammatory immune response, including where the inflammatory response involves Th-17 cells.

nIgM Therapy does not Affect Islet Function

Incubation of mouse or human islets in vitro with 2 and 8 ug nIgM did not affect insulin secretion in response to glucose challenge compared to controls, in glucose-stimulated insulin secretion assay. The stimulation index, for 2 and 8 ug nIgM vs. BSA is 1.9 and 2.0 vs. 1.9 in mice and 2.2 and 1.9 vs. 1.8 in humans. These results indicate that islets remain viable and functional in the presence of nIgM.

### Example 1

### Discussion

Immunomodulatory strategies to prevent or arrest autoimmune T1D are aimed at reversing immune autoreactivity and restoring beta cell mass [22]. The former has mainly focused on targeting T cells and includes the use of, amongst others, monoclonal antibodies specific to membrane expressed receptors such as CD3, CD4, CD40L and B7-2 [23-24, 16-17]; antibodies that interfere with antigen recognition (anti-class II, anti-TCR [25-26]), cellular activation (anti-B7) [17], homing to the pancreas (anti-L selectin and anti-VLA-4) [27] as well as antibodies that target proinflammatory cytokines related to Th1 activity (anti-IFNgamma, anti-TNFalpha, and anti-IL-12) [28]. These strategies have met with limited success, providing transient protection and effective mostly when initiated in the early onset stage of disease development. Clinical islet transplantation represents a definitive intervention in patients diagnosed with T1D [2-4]. However, the requisite long-term use of chemical immunosuppressants, corticosteroids and anti-inflammatory agents that are associated with deleterious side-effects linked to drug toxicity and increased risk of infections and tumors, as well as the transient nature of protection, are major hindrances for its wide spread applicability. Our study using polyclonal serum nIgM was aimed at harnessing the power of innate immunity by using a benign, naturally occurring immune regulatory mechanism to reprogram the immune system to induce the development of beta cell protection and mitigate inflammation, in the absence of chronic immunosuppression.

The NOD mouse has been used extensively to study the many features of the disease shared with human T1D [29]. The disease begins 4-5 weeks after birth, and the initial phase is characterized by a silent and nondestructive infiltration of the perivascular and periductular regions in the pancreas as well as the peripheral islet regions by a heterogenous mixture of CD4 and CD8 T cells, B cells, macrophages and DCs (peri-insulitis) [4, 30]. In the invasive phase that begins at 8-12 weeks of age, the immune infiltrate enters the islet (insulitis) inducing apoptotic beta cell destruction. Significant destruction first becomes evident around 12-13 weeks of age, with mice exhibiting overt diabetes [30-31].

We have demonstrated that nIgM therapy intervention initiated in the early onset stage of disease development prevented disease. Based on our previous published results [7-10], we postulate that this abrogation of autoimmunity might be attributable to nIgM-ALA induced interruption of costimulatory CD28/CD86 signaling, inhibition of proinflammatory cytokine production and chemokine binding, as well as inhibition of autoreactive T cell activation/proliferation. This is supported by our present data wherein nIgM: a) significantly delayed the progression of diabetes development in NOD scid recipients of adoptively transferred CD3 T cells obtained from overtly diabetic NOD mice, indicating that nIgM may act by binding to membrane receptors on autoreactive T cells; and b) inhibited the production of proinflammatory cytokines from NOD splenocytes in vitro. It is conceivable that nIgM-mediated inhibition of autoimmune effector cells allows a slow but continuous increase in beta-cell mass by means of beta-cell neogenesis in the absence of beta-cell apoptosis, a view supported by our histological studies that demonstrated a lack of insulitis, some peri-insulitis and numerous big islets in close proximity to ducts, a process classically observed in beta cell neogenesis, in pancreatic sections obtained from nIgM treated non diabetic mice. We have also demonstrated ˜73% of mice remained normoglycemic at 22-28 weeks post-discontinuation of nIgM therapy, indicating nIgM mediated immunomodulation resulting in the induction of beta cell directed immune ignorance, a significant result with attractive therapeutic potential. Our preliminary flow cytometry data (unpublished) indicated higher CD4+CD25+FoxP3+ expression levels in splenocytes obtained from nIgM-treated non-diabetic NOD mice compared to controls. This observation gains significance when compared to other studies where new onset T1D or cyclophosphamide-induced T1D was associated with a reduction in CD4+CD25+Foxp3+ regulatory T cells [32-33] and where diabetes could be prevented by transfer of CD4+CD25+ regulatory T cells [33]. Detailed flow cytometric analyses of immune cells in nIgM-treated mice that have remained long-term normoglycemic following discontinuation of therapy as well as in vivo experiments involving adoptive transfer of tolerance from these mice into NOD scid mice are underway.

Interestingly, most existing immunosuppressive strategies are unable to prevent diabetic disease progression when administered at >10 weeks of age or are unable to reverse disease after establishment of hyperglycemia [16-17]. Limited data is available on treatments administered in the prehyperglycemic, late-onset phase of disease development (i.e. replicating the clinical scenario where euglycemic patients demonstrate elevated T1D-related auto-antibody levels) aimed at suppressing progressive beta cell destruction. Our studies found a significant reduction in the incidence of diabetes (20%) when nIgM therapy was initiated at 10-11 weeks of age, a point in time in the later stages of beta cell destruction. It is possible that in the 20% of mice that did become diabetic, nIgM administration at the low dosage used (50 ugs nIgM biweekly) may not have succeeded in completely inhibiting autoreactive T cells in the presence of advanced insulitis. This perspective is supported by the fact that treatment with a higher dose (75 ug nIgM administered triweekly) was able to completely restore normoglycemia in mildly diabetic NOD mice with BG in the range of 250-340 mg/dL. nIgM therapy might also induce a new population of regulatory T cells that can lead to a reversal of overt diabetes. Since autoimmune T1D is a disease that is associated with a loss of T cell tolerance leading to the activation, differentiation, and pathogenicity of autoreactive T cells, that nIgM therapy inhibits disease when applied at the latter stages of disease development as well as following establishment of disease (albeit only in the mildly diabetic NOD mice when administered alone), in the absence of chronic immunosuppression is significant.

Optimal T cell response initiation requires costimulatory molecule signaling, in the absence of which T cells perceiving Ag become anergic [34]. It is now known that OX40/OX40L costimulatory molecule expression on T cells in inflamed tissue as well as on activated APCs, e.g., CD11c+CD11b+DCs, coincides late in the lifespan of NOD mice (at >11 or more weeks of age), thereby representing a late checkpoint in T1D development [35]. OX40 signaling results in T cell proliferation, proinflammatory cytokine production, and augmentation of Th17 cell function [36-37]. OX40 is also upregulated on majority of primed “memory” T cells after Ag reencounter. Targeting OX40/OX40L interactions at late onset significantly reduces the incidence of T1D in NOD mice [35]. It is possible that nIgM acts by downregulating expression of these costimulatory molecules on immune cells similar to its down regulation of CD86, CD4 and CD2, thereby interrupting signaling and inhibiting autoreactive T cell activation and proliferation at this later checkpoint in the progression of T1D.

Several groups have associated elevated Th17 cells and IL-17 expression with spontaneous T1D in NOD mice [19-20]. In fact, neutralization of IL-17 post-initiation of insulitis was shown to prevent the development of diabetes, suggesting interference at the effector phase of T1D [21]. Our in vitro results also indicate inhibition of proinflammatory TNF-alpha, IFN-gamma, and IL-17A secretion from NOD mouse splenocytes. We have previously shown that nIgM decreases the frequency of Th17 and CD4 T cells in inflamed tissue and inhibits proinflammatory cells from proliferating and producing IFN-gamma and IL-17 in response to alloantigens, anti-CD3, and a-galactosyl ceramide [7-10]. Tregs were unable to inhibit both Th17 cell activation and IFN-gamma/IL-17 production induced by LPS or in an MLR assay indicating that inhibition was nIgM specific. Taken together, these data highlight the importance of nIgMs and nIgM-ALAs in providing another mechanism to regulate excess inflammation mediated by innate and adaptive immune mechanisms, including IL-17—producing cells.

Preclinical and clinical studies indicate that innate host inflammatory responses occurring in the microenvironment of the graft mediate the early loss of approximately half of the transplanted islet mass in the peritransplant period [15, 38]. These innate inflammatory responses likely promote alloantigen presentation, thereby strengthening and accelerating subsequent cell-mediated allogeneic immune responsiveness. Th-1 and Th-17 subsets produce proinflammatory cytokines, which activate infiltrating leukocytes to mediate the allograft rejection. Our studies have demonstrated that low dose nIgM therapy was able to promote graft survival both in syngeneic and allogeneic islet transplantation. We are currently investigating if combining low dose nIgM therapy with transient immunosuppression or with agents that promote beta cell regeneration, or by increasing the dosage of nIgM to 175 ug nIgM biweekly (which would elevate nIgM serum levels by 150-200 μg/ml) would result in permanent islet allograft survival. In our previous cardiac allo-transplantation experiments, high dose nIgM therapy was able to inhibit severe and rapid BALB/c cardiac allograft rejection in wild type Bl6 recipients [10]. Control C57Bl6 recipients rejected cardiac allografts by 5-7 days with histology demonstrating abundant TH-17 cells, while recipients receiving nIgM had none/minimal rejection. Furthermore, a significant lack of infiltrating leukocytes in the cardiac parenchyma of nIgM-treated recipients decreased CXCL1 production and none or minimal fragmentation of capillaries suggested that nIgM mediated its protective effect by inhibiting processes involved in leukocyte activation and migration. Replenishing IgM in IgM knockout mice with intact B cells and regulatory T cells significantly attenuated inflammation associated with ischemic reperfusion injury and inhibited cardiac allograft rejection, processes involving IFN-g and IL-17. We have also shown that nIgM can directly inhibit Th-17 differentiation and prevent Foxp3+Tregs from becoming proinflammatory under Th-17—inflammatory conditions [10]. Taken together, elevated nIgM-ALA levels could, alone or in the presence of Tregs, attenuate severe inflammatory processes by inhibiting Th-1 and Th-17 proliferation and differentiation and promote allograft survival.

To conclude, our studies indicate that purified polyclonal serum nIgM therapy may represent an innate, effective, safe and benign therapeutic interventional agent, that in the future, may be effectively used to preemptively treat high risk patients who might develop T1D, those with elevated T1D related auto-antibodies or in conjunction with islet transplantation. Our observations also highlight the clinical potential of nIgM therapy in promoting islet graft survival following allotransplantation by regulating excess inflammation mediated by both innate and adaptive immune mechanisms and where the inflammatory responses involves Th-17 cells that are not effectively regulated by regulatory T cells. Additionally, through possible induction of beta cell specific unresponsiveness, nIgM therapy may also prevent recurrence of the disease following islet transplantation. As no generalized immunosuppression was observed with nIgM therapy, it appears to be a safe, naturally occurring, interventional agent for long-term continuous administration without complications usually associated with chronic use of immunosuppressive drugs to prevent T1D.

### Example 2—

### Polyclonal Serum IgM Therapy Promotes the Induction of n-Cell Specific Hyporesponsiveness in the Non-Obese Diabetic Mouse Model of Type 1 Diabetes

Purpose:

To investigate if purified polyclonal serum IgM therapy promotes the induction of β-cell specific hypo-responsiveness (tolerance) in the non-obese diabetic (NOD) mouse model of autoimmune type 1 diabetes (T1D). To study this, an “adoptive transfer of T1D disease” model as well as an “adoptive transfer of β-cell specific tolerance” model was used wherein immunodeficient NOD scid mice were used as recipients of immune cells obtained from diabetic and/or tolerant mice following which the development of hyperglycemia was evaluated.

Methods:

In experiment #1, 5-6 wks-old immunodeficient NOD scid mice served as adoptive transfer recipients of 2×10⁷ splenocytes or 7.5×10⁶ CD3 T-cells IP from diabetic mice or from 2-3 wks-old prediabetic NOD mice. Mice were divided into 75 μg biweekly IgM therapy-receiving test group and BSA-injected control group. In experiment #2, 1×10⁷ splenocytes from diabetic NOD mice (n=4) or IgM treated non-diabetic β-cell specific hyporesponsive (tolerant) NOD mice (n=5) were injected intra-peritoneal either alone or combined in 1:1 (n=5), 1:2 (n=3) or 1:3 (n=3) ratio. IgM treated tolerant mice were mice that remained non-diabetic for >25 weeks post-IgM therapy discontinuation. Mice with blood glucose level 250 mg/dL on 3 successive days were considered diabetic.

Results:

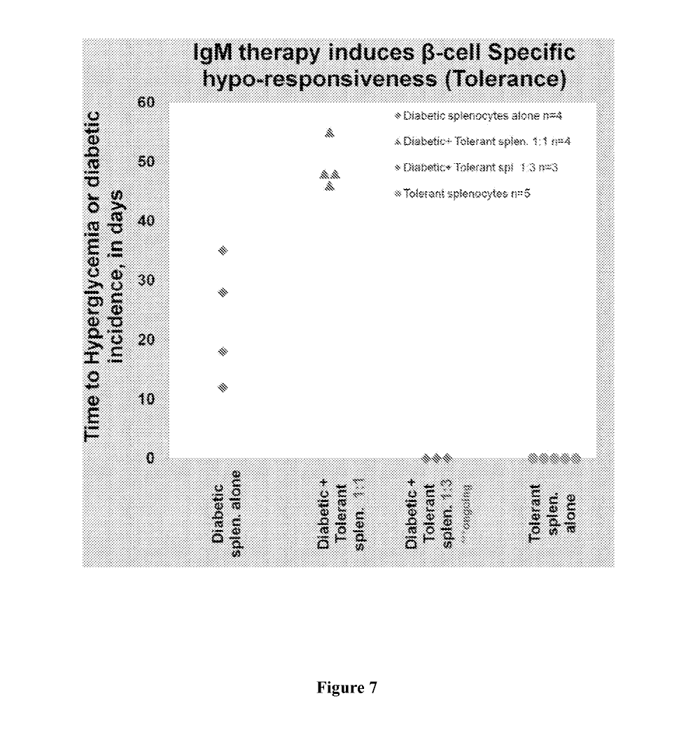

Adoptive transfer of NOD CD3 T cells or splenocytes from diabetic NOD mice into 5-6 wks old NOD scid recipients resulted in hyperglycemia. The appearance of diabetes was significantly delayed in NOD scid mice receiving IgM therapy (53.0±19.1 days; n=7) when compared with the control group (24.0±1.0 days; n=7) that received PBS/BSA (P<0.001). Results were similar with splenocytes or CD3 T cells. Recipients of splenocytes/CD3 T cells from nondiabetic 2-3 wk old NOD mice did not become diabetic. Recipients of splenocytes from tolerant mice alone remained permanently non-diabetic. When 1×107 splenocytes from diabetic NOD mice were transferred along with splenocytes from IgM-treated tolerant mice, no transfer of disease was observed as long as the diabetic to tolerant splenocyte ratio was 1:2, i.e., splenocytes from tolerant mice were twice the number as those from diabetic mice. 1:1 ratio however resulted in significant delay of the appearance of hyperglycemia (p<0.001). (See FIG. 7).

Conclusion:

IgM therapy promotes the induction of β-cell specific hypo-responsiveness in the NOD mouse model of T1D. The use of purified polyclonal serum IgM antibody therapy has significant clinical translational potential in the cure of T1D.

### Example 3—

### The Effect of Polyclonal IgM Antibody Therapy on Mouse Insulin Autoantibody (m-IAA) Levels at Progressive Stages of Type 1 Diabetes

Purpose:

The diabetes-prone, non-obese diabetic (NOD) mouse is a murine model for human autoimmune type 1 diabetes (T1D). The invasive phase of the disease (at 8-12 weeks of age) is characterized by ‘insulitis’, a silent, nondestructive immune cell infiltration of the islets that induces apoptotic β-cell destruction, eventually resulting in the development of hyperglycemia (marking the establishment of overt diabetes) at around 12-13 weeks of age The development of T1D pathogenesis is characterized by the presence of anti-islet autoantibodies such as GAD65 autoantibody (GADA), islet cell autoantibodies (ICA), insulinoma associated-2 autoantibodies (IA-2A) and insulin autoantibodies (IAA). Elevated levels of 2 or more of these autoantibodies are considered a marker for predisposition to islet cell specific autoimmunity Studies indicate that high titers and the early appearance of IAAs are associated with an early age of disease onset. Herein, we studied the effect of purified polyclonal IgM therapy on mouse IAA levels at different stages of the disease.

Methods:

Cross-sectional evaluation of m-IAA levels was conducted using plasma samples obtained from female NOD/ShiltJ mice (Jackson Laboratories, Bar Harbor, Me.) in the following categories: non-diabetic mice (2-5 weeks old, n=3), prehyperglycemic mice (8-12 weeks old, n=1), diabetic mice (14-22 weeks old, n=5) and IgM treated mice (parallel to prehyperglycemic and diabetic age groups, n=11). Blood glucose levels (nonfasting) were monitored daily (AccuChek Aviva glucometer, Roche) and diabetes was defined as non-fasting plasma blood glucose (BG)≧250 mg/dl. Insulin autoantibody (IAA) was measured by a micro-IAA assay (mIAA) at the Barbara Davis Center for Diabetes, Colorado. Briefly, 125-I labeled insulin (Amersham) was incubated with 5 ul of plasma with and without cold insulin and the immune complex precipitated with protein A and G Sepharose. Radioactivity was counted on a Topcount 96-well plate beta counter. An index was calculated based upon delta cpm between wells without and with cold insulin, and was expressed as index=(sample Δ cpm−negative control Δ cpm)/(positive control Δ cpm−negative control Δ cpm), with a positivity criterion of 0.010.

Results:

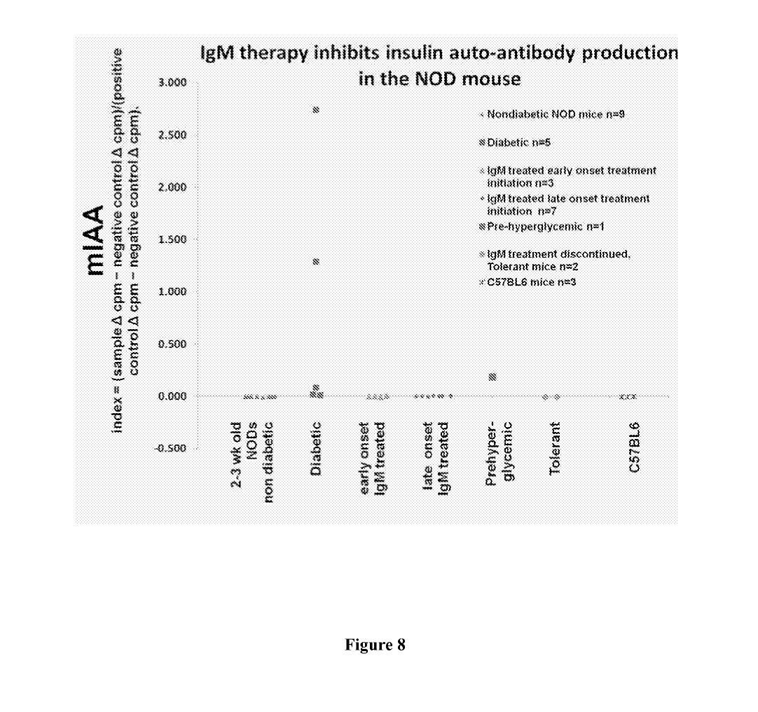

Preliminary results indicate that m-IAA levels (Index) were elevated in mice belonging to the prehyperglycemic group and the diabetic group. m-IAA levels in the diabetic group (n=5) were either very high (2/5), moderate (1/5) or very low (2/5), while they were moderate in the prehyperglycemic group (n=1). The m-IAA levels in the IgM treated group were zero, similar to the control non-diabetic group. In mice in which IgM treatment had been discontinued and remained non-diabetic ˜25 week post-discontinuation of therapy, the m-IAA levels were zero. (See FIG. 8).

Conclusion:

mIAA production marking the onset and progression of T1D disease pathogenesis is markedly inhibited by IgM therapy, indicating the beneficial effect of IgM therapy in prevention of T1D disease development.

### Example 4—

### Polyclonal Serum Immunoglobulin M Antibody Therapy Prevents the Onset and Progression of Type 1 Diabetes, Induces the Development of n-Cell Specific Hyporesponsiveness and Promotes Islet Transplantation Outcomes

Purpose:

To study the effect of polyclonal serum Immunoglobulin M (IgM) therapy in preventing the onset and progression of type 1 diabetes (T1D), inducing β-cell specific hyporesponsiveness (tolerance) and promoting islet transplantation.

Methods:

4-5 wks-old non-obese diabetic (NOD) mice received either 75 μg IgM therapy biweekly or PBS/BSA intraperitoneal (IP) until 18 weeks of age, and blood glucose (BG) levels monitored. In adoptive transfer of tolerance or of disease experiments, 5-6 wks-old NOD-scid mice served as recipients of 1-2×10⁷ splenocytes or 7.5×10⁶ CD3 T-cells IP from tolerant mice (mice that remained non-diabetic for >25 weeks post-IgM therapy discontinuation) and/or from diabetic mice, with or without IgM therapy. Plasma insulin autoantibody (IAA) levels were measured by radioimmunoassay. 10-12 weeks-old female BALB/c mice served as donors in islet allotransplantation and retired female C57BL/6 mice in syngeneic transplantation experiments.

Results:

When IgM therapy was initiated at 4-5 weeks of age, none of the IgM-treated mice (n=33) became diabetic. In contrast, 80-90% control mice (n=30) became diabetic by 18-20 weeks of age. Initiated at 11 weeks of age, only 20% (n=20) mice became diabetic (p<0.0001). Discontinuing therapy resulted in hyperglycemia in only 27% mice at 28 weeks post-discontinuation, indicating induction of β-cell specific hyporesponsiveness in the remaining animals. IAA production, a marker for the onset/progression of T1D pathogenesis, was significantly inhibited by IgM therapy (p<0.001). IAA in the diabetic group (n=5) was either very high (2/5), moderate (1/5) or low (2/5), and moderate in the prehyperglycemic group (n=1). In contrast, IAA levels in the IgM-treated group (n=10) and tolerant mice (n=2) were 0. Adoptive transfer of CD3 T-cells from diabetic NOD mice into NOD-scid recipients resulted in hyperglycemia in 24.0±1.0 days (n=7), which was significantly delayed by IgM therapy (53.0±19.1 days; n=7) (p<0.001). Recipients of splenocytes from tolerant mice alone (n=5) remained permanently non-diabetic. When diabetic to tolerant splenocytes ratios were 1:1 (n=5) and 1:2 (n=3), the incidence of hyperglycemia was significantly delayed, while with 1:3 ratio (n=3), mice have remained non-diabetic. The mean survival time of BALB/c islet allografts was 41.2±3.3 days for IgM-treated C57BL6 recipients (n=4, fifth remained normoglycemic) vs. 10.2±2.6 days for controls (n=5, p<0.001). In syngeneic transplantation, time to return to normoglycemia was 15.4±3.6 days for IgM-treated recipients (n=5) and >35 days for controls (n=4).

Conclusion:

IgM therapy prevents the onset and progression of T1D, induces β-cell specific hyporesponsiveness, and promotes islet transplantation. IgM therapy has significant clinical translational potential in the prevention and cure of T1D and in promoting islet transplantation outcomes.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

1. Yoon J W, Jun H S. Autoimmune destruction of pancreatic beta cells. Am J Ther 2005; 12:580-591.
2. Huang X, Moore D J, Ketchum R J, et al. Resolving the conundrum of islet transplantation by linking metabolic dysregulation, inflammation, and immune regulation. Endocr Rev 2008; 29:603-630.
3. Jahansouz C, Kumer S C, Ellenbogen M et al. Evolution of 13-Cell Replacement Therapy in Diabetes Mellitus: Pancreas Transplantation. Diabetes Technol Ther 2011; 13:395-418.
4. Chhabra P, Brayman K L. Current status of immunomodulatory and cellular therapies in preclinical and clinical islet transplantation. J Transplant 2011; 2011:637692.
5. Mineo D, Sageshima J, Burke G W, Ricordi C. Minimization and withdrawal of steroids in pancreas and islet transplantation. Transpl Int 2009; 22:20-37.
6. Rother K I, Harlan D M. Challenges facing islet transplantation for the treatment of type 1 diabetes mellitus. J Clin Invest 2004; 114:877-883.
7. Lobo P I, Schlegel K H, Spencer C E, et al. Naturally occurring IgM anti-leukocyte autoantibodies (IgM-ALA) inhibit T cell activation and chemotaxis. J Immunol 2008; 180:1780-1791.
8. Lobo P I, Schlegal K H, Vengal J, et al. Naturally occurring IgM anti-leukocyte autoantibodies inhibit T-cell activation and chemotaxis. J Clin Immunol 2010; 30:S31-36.
9. Lobo P I, Schlegel K H, Yuan W, et al. Inhibition of HIV-1 infectivity through an innate mechanism involving naturally occurring IgM anti-leukocyte autoantibodies. J Immunol 2008; 180:1769-1779.
10. Lobo P I, Bajwa A, Schlegel K H, et al. Natural IgM anti-leukocyte autoantibodies attenuate excess inflammation mediated by innate and adaptive immune mechanisms involving Th-17. J Immunol 2012; 188:1675-1785.
11. Kerman R H, Susskind B, Buyse I, et al. Flow cytometry-detected IgG is not a contraindication to renal transplantation: IgM may be beneficial to outcome. Transplantation 1999; 68:1855-1858.
12. Przybylowski P, Balogna M, Radovancevic B, et al. The role of flow cytometry-detected IgG and IgM anti-donor antibodies in cardiac allograft recipients. Transplantation 1999; 67:258-262.
13. Boes M. Role of natural and immune IgM antibodies in immune responses. Mol Immunol 2000; 37:1141-1149.
14. Manson J J, Mauri C, Ehrenstein M R. Natural serum IgM maintains immunological homeostasis and prevents autoimmunity. Springer Semin Immunopathol 2005; 26:425-432.
15. Chhabra P, Wang K, Zeng Q, Jecmenica M, et al. Adenosine A(2A) agonist administration improves islet transplant outcome: Evidence for the role of innate immunity in islet graft rejection. Cell Transplant 2010; 19:597-612.
16. Balasa B, Krahl T, Patstone G, et al. CD40 ligand-CD40 interactions are necessary for the initiation of insulitis and diabetes in nonobese diabetic mice. J Immunol 1997; 159:4620-4627.
17. Lenschow D J, Ho S C, Sattar H, et al. Differential effects of anti-B7-1 and anti-B7-2 monoclonal antibody treatment on the development of diabetes in the nonobese diabetic mouse. J Exp Med 1995; 181:1145-1155.
18. Christianson S W, Shultz L D, Leiter E H. Adoptive transfer of diabetes into immunodeficient NOD-scid/scid mice. Relative contributions of CD4+ and CD8+ T-cells from diabetic versus prediabetic NOD.NON-Thy-1a donors. Diabetes 1993; 42:44-55.
19. Emamaullee, J A, Davis J, Merani S, et al. Inhibition of Th17 cells regulates autoimmune diabetes in NOD mice. Diabetes 2009; 58:1302-1311.
20. Honkanen J, Nieminen J K, Gao R, et al. IL-17 immunity in human type 1 diabetes. J Immunol 2010; 185:1959-1967.
21. Jain R, Tartar D M, Gregg R K, et al. Innocuous IFNgamma induced by adjuvant-free antigen restores normoglycemia in NOD mice through inhibition of IL-17 production. J Exp Med 2008; 205: 207-218.
22. Boettler T, von Herrath M. Immunotherapy of type 1 diabetes—how to rationally prioritize combination therapies in T1D. Int Immunopharmacol 2010; 10:1491-1495.
23. Chatenoud L, Thervet E, Primo J, et al. Anti-CD3 antibody induces long-term remission of overt autoimmunity in nonobese diabetic mice. Proc Nat. Acad Sci USA 1993; 91, 123-127.
24. Shizuru J A, Taylor-Edwards C, Banks B A, et al Immunotherapy of the nonobese diabetic mouse; treatment with an antibody to T helper lymphocytes. Science 1988:240, 659-662.
25. Boitard C, Bendelac A, Richard M F, et al. Prevention of diabetes in nonobese diabetic mice by anti-I-A monoclonal antibodies: transfer of protection by splenic T cells. Proc Natl Acad Sci USA 1988; 85: 9719-9723.
26. Sempe P, Bedossa P, Richard, M F, et al. Anti-a/b T cell receptor monoclonal antibody provides an efficient therapy for autoimmune diabetes in nonobese diabetic (NOD) mice. Eur J Immunol 1991; 21:1163-1169.
27. Yang X D, Karin N, Tisch R, et al. Inhibition of insulitis and prevention of diabetes in nonobese diabetic mice by blocking L-selectin and very late antigen 4 adhesion receptors. Proc Natl Acad Sci USA 1993; 90:10494-10498.
28. Rabinovitch A Immunoregulatory and cytokine imbalances in the pathogenesis of IDDM. Therapeutic intervention by immunostimulation. Diabetes 1994; 43:613-621.
29. Chaparro R J, Dilorenzo T P. An update on the use of NOD mice to study autoimmune (Type 1) diabetes. Expert Rev Clin Immunol 2010; 6:939-955.
30. Tisch R, McDevitt H. Insulin-dependent diabetes mellitus. Cell 1996; 85:291-297.
31. Pakala S V, Bansal-Pakala P, Halteman B S, et al. Prevention of diabetes in NOD mice at a late stage by targeting OX40/OX40 ligand interactions. Eur J Immunol 2004; 34:3039-3046.
32. Takiishi T, Korf H, Van Belle T L, et al. Reversal of autoimmune diabetes by restoration of antigen-specific tolerance using genetically modified Lactococcus lactis in mice. J Clin Invest 2012 Apr. 9. pii: 60530. doi: 10.1172/JCI60530 [Epub ahead of print]. Accessed on Apr. 19, 2012. Available at: http://www.jci.org/articles/view/60530#B22.
33. Brode S, Raine T, Zaccone P, et al. Cyclophosphamide-induced type-1 diabetes in the NOD mouse is associated with a reduction of CD4+CD25+Foxp3+ regulatory T cells. J Immunol 2006; 177:6603-6612.
34. Snanoudj R, de Preneuf H, Creput C, et al. Costimulation blockade and its possible future use in clinical transplantation. Transpl Int 2006; 19:693-704.
35. Pakala S V, Bansal-Pakala P, Halteman B S, et al. Prevention of diabetes in NOD mice at a late stage by targeting OX40/OX40 ligand interactions. Eur J Immunol 2004; 34:3039-3046.
36. Ishii N, Takahashi T, Soroosh P, Sugamura K. OX40-OX40 ligand interaction in T-cell-mediated immunity and immunopathology. Adv Immunol 2010; 105:63-98.
37. Zhang Z, Zhong W, Hinrichs D, et al. Activation of OX40 augments Th17 cytokine expression and antigen-specific uveitis. Am J Pathol 2010; 177:2912-2920.
38. Jahansouz C, Jahansouz C, Kumer S C, et al. Evolution of β-Cell Replacement Therapy in Diabetes Mellitus: Islet Cell Transplantation. J Transplant 2011; 2011:247959.

What is claimed is:

1. A method of preventing or treating Type 1 Diabetes, said method comprising administering to a subject in need thereof a pharmaceutical composition comprising an effective amount of naturally occurring IgM (nIgM), a pharmaceutically-acceptable carrier, and optionally at least one additional therapeutic agent.

2. The method of claim 1, wherein said nIgM is polyclonal serum nIgM.

3. The method of claim 2, wherein said nIgM is purified.

4. The method of claim 1, wherein said nIgM is purified by column chromatography and then concentrated.

5. The method of claim 4, further wherein said purified nIgM is dialyzed against a pharmaceutical composition, buffer, or medium and filter sterilized.

6. The method of claim 1, wherein said nIgM is obtained from said subject.

7. The method of claim 1, wherein said nIgM is administered at a dose of about 0.01 mg nIgM/kg body weight to about 30 mg nIgM/kg body weight.

8. The method of claim 7, wherein said nIgM is administered at a dose of about 0.1 mg nIgM/kg body weight to about 25 mg nIgM/kg body weight.

9. The method of claim 8, wherein said nIgM is administered at a dose of about 1.0 mg nIgM/kg body weight to about 20 mg nIgM/kg body weight.

10. The method of claim 1, wherein said nIgM is administered at least twice.

11. The method of claim 1, wherein said pharmaceutical composition is administered by a method selected from intravenously, intraperitoneally, and intraarterially.

12. The method of claim 1, wherein said pharmaceutical composition is administered up to about 5 times.

13. The method of claim 1, wherein said pharmaceutical composition is administered up to about 50 times.

14. The method of claim 1, wherein said pharmaceutical composition is administered at least about 50 times.

15. The method of claim 1, wherein at least one of said therapeutic agents is a cell.

16. The method of claim 15, wherein said cell is selected from the group consisting of cord blood cells, islet cells, dendritic cells, regulatory T cells, stem cells, insulin-producing cells, mesenchymal stem cells, induced pluripotent stem cells, embryonic stem cells, hematopoietic stem cells, adipocyte stem cells, and neural stem cells.

17. The method of claim 1, wherein said pharmaceutical composition is administered beginning in the early or late onset stage of prehyperglycemic beta cell destruction.

18. The method of claim 1, wherein said method inhibits insulitis and promotes beta cell neogenesis.

19. The method of claim 1, wherein said method induces beta cell specific hyporesponsiveness.

20. The method of claim 1, wherein said method inhibits insulin autoantibody production.

21. The method of claim 1, wherein said method inhibits beta cell destruction.

22. The method of claim 1, wherein said method inhibits periductular/perivascular inflammation in the pancreas.

23. The method of claim 1, wherein said at least one additional therapeutic agent is selected from the group consisting of anti-microbial agents, anti-inflammatory agents, anesthetic agents, analgesic agents, steroids, glucagon-like peptide 1 receptor agonists, dipeptidyl peptidase IV inhibitors, and immunodulatory agents.

24. A method of enhancing survival of an islet or pancreatic graft in a subject, said method comprising administering to a subject in need thereof a pharmaceutical composition comprising an effective amount of naturally occurring IgM (nIgM), a pharmaceutically-acceptable carrier, and optionally at least one additional therapeutic agent.

25. The method of claim 24, wherein said method restores normoglycemia in a Type 1 Diabetic.

26. The method of claim 24, wherein said method does not affect islet function.

27. The method of claim 24, wherein said nIgM is administered before said graft is transplanted.

28. The method of claim 24, wherein said nIgM is administered after said graft is transplanted.

29. The method of claim 24, wherein said nIgM is administered before and after said graft is transplanted.

30. The method of claim 24, wherein said nIgM is polyclonal serum nIgM.

31. The method of claim 30, wherein said nIgM is purified.

32. The method of claim 31, wherein said purified nIgM is purified from blood or serum by column chromatography and then concentrated.

33. The method of claim 32, further wherein said purified nIgM is dialyzed against a pharmaceutical composition, buffer, or medium and filter sterilized.

34. The method of claim 24, wherein said nIgM is obtained from said subject.

35. The method of claim 24, wherein said nIgM is administered at a dose of about 0.01 mg nIgM/kg body weight to about 30 mg nIgM/kg body weight.

36. The method of claim 35, wherein said nIgM is administered at a dose of about 0.1 mg nIgM/kg body weight to about 25 mg nIgM/kg body weight.

37. The method of claim 36, wherein said nIgM is administered at a dose of about 1.0 mg nIgM/kg body weight to about 20 mg nIgM/kg body weight.

38. The method of claim 24, wherein said nIgM is administered at least twice.

39. The method of claim 24, wherein said pharmaceutical composition is administered by a method selected from intravenously, intraperitoneally, and intraarterially.

40. The method of claim 24, wherein said pharmaceutical composition is administered up to about 5 times.

41. The method of claim 24, wherein said pharmaceutical composition is administered up to about 50 times.

42. The method of claim 24, wherein said pharmaceutical composition is administered at least about 50 times.

43. The method of claim 24, wherein at least one of said therapeutic agents is a cell.

44. The method of claim 43, wherein said cell is selected from the group consisting of cord blood cells, islet cells, dendritic cells, regulatory T cells, stem cells, insulin-producing cells, mesenchymal stem cells, induced pluripotent stem cells, embryonic stem cells, hematopoietic stem cells, adipocyte stem cells, and neural stem cells.

45. The method of claim 24, wherein said method inhibits periductular/perivascular inflammation in the pancreas.

46. The method of claim 24, wherein said at least one additional therapeutic agent is selected from the group consisting of anti-microbial agents, anti-inflammatory agents, anesthetic agents, analgesic agents, steroids, glucagon-like peptide 1 receptor agonists, dipeptidyl peptidase IV inhibitors, and immunodulatory agents.

47. The method of claim 24, wherein said subject has Type 1 Diabetes.

48. A kit for use in preventing or treating Type 1 Diabetes or for enhancing islet graft survival, said kit comprising at least one dose of nIgM or source of nIgM, optionally at least one additional therapeutic agent, an applicator, and an instructional material for the use thereof.
